(19) United States
(12) Patent Application Publication
(10) Pub. No.: US 2023/0103724 A1
(43) Pub. Date: Apr. 6, 2023

(54) SOLID STATE FORMS OF AVAPRITINIB AND PROCESS FOR PREPARATION THEREOF

(71) Applicant: TEVA CZECH INDUSTRIES S.R.O, Opava-Komarov (CZ)

(72) Inventors: Anantha Rajmohan Muthusamy, Sivakasi (IN); Amit Singh, Greater Noida (IN); Sundara Lakshmi Kanniah, Vellore (IN); Vaibhav Suresh Daund, Nasik (IN); Parven Kumar Luthra, Noida (IN); Yogesh Dhananjay Wagh, Badlapur (E) Thane (IN); Meenakshi Sundaram Somasundaram, Virudu Nagar Dt (IN)

(21) Appl. No.: 17/908,286

(22) PCT Filed: Mar. 11, 2021
(86) PCT No.: PCT/US21/21809

§ 371 Date: Aug. 31, 2022

(30) Foreign Application Priority Data
Mar. 11, 2020 (IN) 202011010365
Dec. 11, 2020 (IN) 202011053964

(51) Int. Cl. C07D 487/04

(52) U.S. Cl. CPC C07D 487/04; C07B 2200/13

(57) ABSTRACT

The present disclosure encompasses solid state forms of Avapritinib, in embodiment crystalline polymorphs of Avapritinib, processes for preparation thereof, and pharmaceutical compositions thereof.

### FIELD OF THE DISCLOSURE

The present disclosure encompasses solid state forms of Avapritinib, in embodiment crystalline polymorphs of Avapritinib, processes for preparation thereof, and pharmaceutical compositions thereof.

### BACKGROUND OF THE DISCLOSURE

Avapritinib, (1S)-1-(4-fluorophenyl)-1-[2-[4-[6-(1-methylpyrazol-4-yl)pyrrolo[2,1-f][1,2,4]triazin-4-yl]piperazin-1-yl]pyrimidin-5-yl]ethanamine, has the following chemical structure:

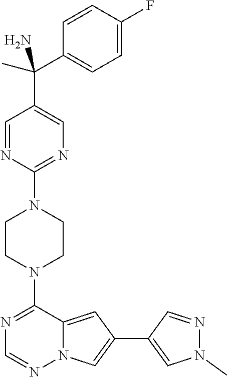 [SMILES: Cn1cc(-c2cc3c(N4CCN(c5ncc([C@@](C)(N)c6ccc(F)cc6)cn5)CC4)ncnn3c2)cn1]

Avapritinib is being developed for the treatment of gastrointestinal stromal tumors (GIST), solid tumors. Avapritinib is also under evaluation for the treatment of Advanced Systemic Mastocytosis.

The compound is described in International Publication No. WO 2015/057873. International Publication Nos. WO 2020/210669, WO 2021/004895 and Chinese Publication No. CN 112125910A disclose crystalline forms of Avapritinib.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Avapritinib.

### SUMMARY OF THE DISCLOSURE

The present disclosure provides crystalline polymorphs of Avapritinib, processes for preparation thereof, and pharmaceutical compositions thereof. These crystalline polymorphs can be used to prepare other solid state forms of Avapritinib, Avapritinib salts and their solid state forms.

The present disclosure also provides uses of the said solid state forms of Avapritinib in the preparation of other solid state forms of Avapritinib or salts thereof.

The present disclosure provides crystalline polymorphs of Avapritinib for use in medicine, including for the treatment of gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST).

The present disclosure also encompasses the use of crystalline polymorphs of Avapritinib of the present disclosure for the preparation of pharmaceutical compositions and/or formulations.

In another aspect, the present disclosure provides pharmaceutical compositions comprising crystalline polymorphs of Avapritinib according to the present disclosure.

The present disclosure includes processes for preparing the above mentioned pharmaceutical compositions. The processes include combining any one or a combination of the crystalline polymorphs of Avapritinib with at least one pharmaceutically acceptable excipient.

The crystalline polymorphs of Avapritinib as defined herein and the pharmaceutical compositions or formulations of the crystalline polymorphs of Avapritinib may be used as medicaments, such as for the treatment of gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST).

The present disclosure also provides methods of treating gastrointestinal stromal tumors (GIST), by administering a therapeutically effective amount of any one or a combination of the crystalline polymorphs of Avapritinib of the present disclosure, or at least one of the above pharmaceutical compositions, to a subject suffering from gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST), or otherwise in need of the treatment.

The present disclosure also provides uses of crystalline polymorphs of Avapritinib of the present disclosure, or at least one of the above pharmaceutical compositions, for the manufacture of medicaments for treating e.g., gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST).

### BRIEF DESCRIPTION OF THE DRAWINGS

FIG. 1 shows a characteristic X-ray powder diffraction pattern (XRPD) of Avapritinib Form AT1.

FIG. 2 shows a characteristic X-ray powder diffraction pattern (XRPD) of amorphous Avapritinib.

FIG. 3 shows a characteristic X-ray powder diffraction pattern (XRPD) of Avapritinib Form AT2.

FIG. 4 shows a characteristic X-ray powder diffraction pattern (XRPD) of Avapritinib Form AT3.

FIG. 5 shows a characteristic X-ray powder diffraction pattern (XRPD) of Avapritinib Form AT4.

FIG. 6 shows a characteristic X-ray powder diffraction pattern (XRPD) of Avapritinib Form AT5.

FIG. 7 shows a characteristic X-ray powder diffraction pattern (XRPD) of Avapritinib Form AT6.

FIG. 8: Solid state ¹³C NMR spectrum of Avapritinib Form AT4 (full scan)

FIG. 9: Solid state ¹³C NMR spectrum of Avapritinib Form AT4 (0-100 ppm)

FIG. 10: Solid state ¹³C NMR spectrum of Avapritinib Form AT4 (100-200 ppm)

### DETAILED DESCRIPTION OF THE DISCLOSURE

The present disclosure encompasses solid state forms of Avapritinib, including crystalline polymorphs of Avapritinib, processes for preparation thereof, and pharmaceutical compositions thereof.

Solid state properties of Avapritinib and crystalline polymorphs thereof can be influenced by controlling the conditions under which Avapritinib and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Avapritinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Avapritinib. In some embodiments of the disclosure, the described crystalline polymorph of Avapritinib may contain from about 1% to about 20% (w/w), from about 5% to about 20% (w/w), or from about 5% to about 10% (w/w) of one or more other crystalline polymorph of the same Avapritinib.

Depending on which other crystalline polymorphs a comparison is made, the crystalline polymorphs of Avapritinib of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density. The crystalline polymorphs of the present disclosure may, in particular, be stable to stress conditions such as: grinding, pressure, heating, and/or exposure to humidity, and may have advantageous solubility characteristics at physiologically relevant pH values.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Avapritinib referred to herein as being characterized by graphical data “as depicted in” or “as substantially depicted in” a Figure will thus be understood to include any crystal forms of Avapritinib characterized with the graphical data having such small variations, as are well known to the skilled person, in comparison with the Figure.

As used herein, and unless stated otherwise, the term “anhydrous” in relation to crystalline forms of Avapritinib, relates to a crystalline form of Avapritinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Avapritinib of the present disclosure corresponds to a crystalline polymorph of Avapritinib that is physically separated from the reaction mixture in which it is formed.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

As used herein, solid state ¹³C NMR was carried out at room temperature (300K), with spinning frequency of 11 kHz.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The present disclosure includes a crystalline polymorph of Avapritinib, designated AT1. The crystalline Form AT1 of Avapritinib may be characterized by data selected from one or more of the following: an X-ray powder diffraction pattern substantially as depicted in FIG. 1; an X-ray powder diffraction pattern having peaks at 3.8, 16.6, 21.4, 22.8 and 23.7 degrees 2-theta±0.2 degrees 2-theta; and combinations of these data.

Crystalline Form AT1 of Avapritinib may be further characterized by an X-ray powder diffraction pattern having peaks at 3.8, 16.6, 21.4, 22.8 and 23.7 degrees 2-theta±0.2 degrees 2-theta, and also having any one, two, three, four or five additional peaks selected from 11.4, 13.7, 19.9, 25.1 and 30.5 degrees 2-theta±0.2 degrees 2-theta.

In one embodiment of the present disclosure, crystalline Form AT1 of Avapritinib is isolated.

Crystalline Form AT1 of Avapritinib may be characterized by each of the above characteristics alone/or by all possible combinations, e.g., an XRPD pattern having peaks at 3.8, 16.6, 21.4, 22.8 and 23.7 degrees 2-theta±0.2 degrees 2-theta; an XRPD pattern as depicted in FIG. 1, and combinations thereof.

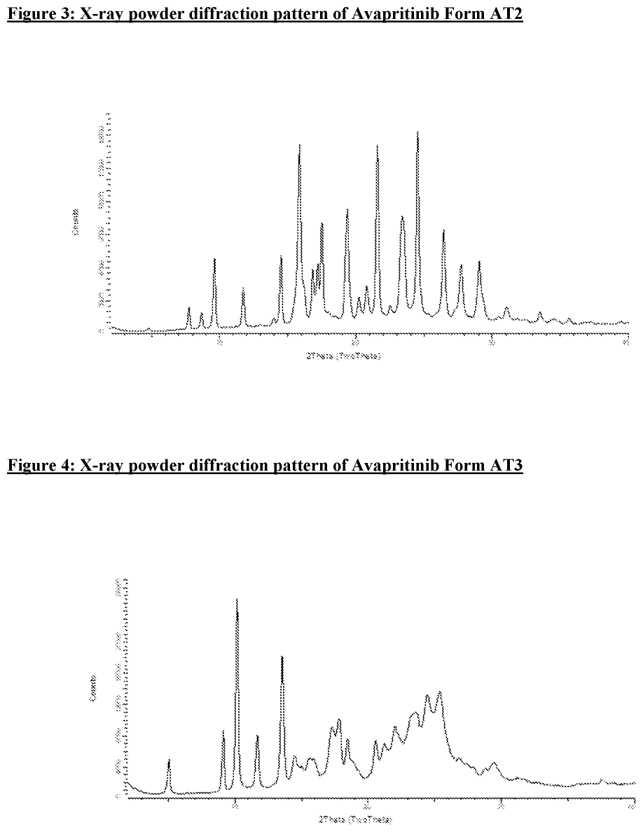

The Present disclosure includes also a crystalline polymorph of Avapritinib, designated AT2. The crystalline Form AT2 of Avapritinib may be characterized by data selected from one or more of the following: an X-ray powder diffraction pattern substantially as depicted in FIG. 3; an X-ray powder diffraction pattern having peaks at 9.6, 15.9, 19.4, 21.6 and 24.5 degrees 2-theta±0.2 degrees 2-theta; and combinations of these data.

Crystalline Form AT2 of Avapritinib may be further characterized by an X-ray powder diffraction pattern having peaks at 9.6, 15.9, 19.4, 21.6 and 24.5 degrees 2-theta±0.2 degrees 2-theta, and also having any one, two, three, four or five additional peaks selected from 11.7, 14.5, 23.4, 26.4 and 29.0 degrees 2-theta±0.2 degrees 2-theta.

In one embodiment of the present disclosure, crystalline Form AT2 of Avapritinib is isolated.

In a further embodiment, Form AT2 of Avapritinib may be a solvate.

Yet in a further embodiment, Form AT2 of Avapritinib may be ethyl formate solvate.

The present disclosure encompasses a crystalline polymorph of Avapritinib, designated AT3. The crystalline Form AT3 of Avapritinib may be characterized by data selected from one or more of the following: an X-ray powder diffraction pattern substantially as depicted in FIG. 4; an X-ray powder diffraction pattern having peaks at 5.1, 9.2, 10.2, 11.7 and 13.5 degrees 2-theta±0.2 degrees 2-theta; and combinations of these data.

Crystalline Form AT3 of Avapritinib may be further characterized by an X-ray powder diffraction pattern having peaks at 5.1, 9.2, 10.2, 11.7 and 13.5 degrees 2-theta±0.2 degrees 2-theta, and also having any one, two, three or four additional peaks selected from 17.8, 18.5, 20.6 and 23.5 degrees 2-theta±0.2 degrees 2-theta.

In one embodiment of the present disclosure, crystalline Form AT3 of Avapritinib is isolated.

Crystalline Form AT3 may be a hydrate.

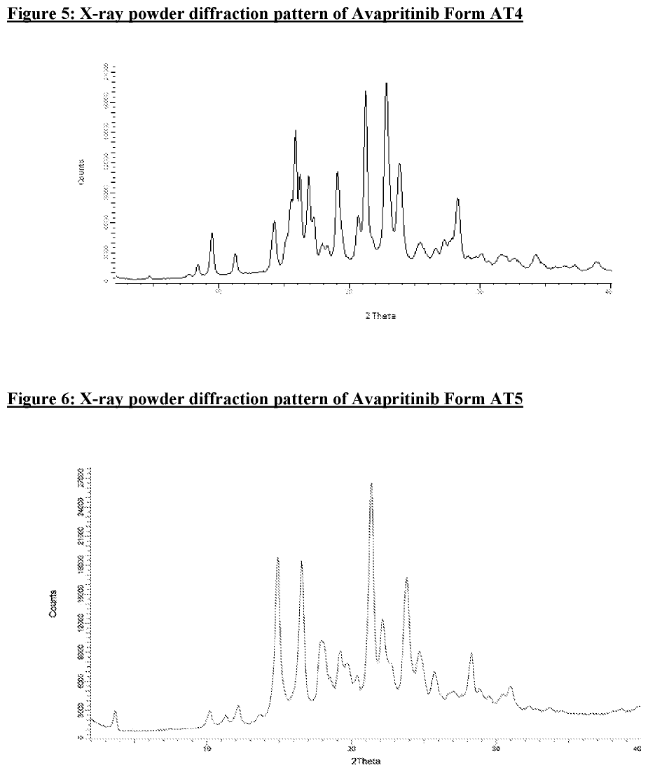

The present disclosure comprises also a crystalline polymorph, designated AT4. The crystalline Form AT4 of Avapritinib may be characterized by data selected from one or more of the following: an X-ray powder diffraction pattern substantially as depicted in FIG. 5; an X-ray powder diffraction pattern having peaks at 11.3, 16.3, 21.3, 22.9 and 23.9 degrees 2-theta±0.2 degrees 2-theta; and combinations of these data.

Crystalline Form AT4 of Avapritinib may be further characterized by an X-ray powder diffraction pattern having peaks at 11.3, 16.3, 21.3, 22.9 and 23.9 degrees 2-theta±0.2 degrees 2-theta, and also having any one, two, three or four additional peaks selected from 9.5, 14.3, 15.9, and 16.9 degrees 2-theta±0.2 degrees 2-theta.

Crystalline Form AT4 of Avapritinib may alternatively be characterized by an XRPD pattern having peaks at 9.5, 11.3, 14.3, 15.9, 16.3, 16.9, 21.3, 22.9 and 23.9 degrees 2-theta±0.2 degrees 2-theta.

Crystalline Form AT4 of Avapritinib may be alternatively or additionally characterized by a solid state ¹³C NMR spectrum with peaks at 27.7, 57.7, 113.2, 123.3, 139.6 and 178.7 ppm±0.2 ppm. Alternatively or additionally, crystalline Form AT4 of Avapritinib may be characterized by a solid state ¹³C NMR spectrum having the following chemical shift absolute differences from a peak at 43.4 ppm±2 ppm of 15.7, 14.3, 69.8, 79.9, 96.2 and 135.3 ppm±0.1 ppm. Optionally, Form AT4 of Avapritinib may be characterized by a solid state ¹³C NMR spectrum substantially as depicted in any of FIG. 8, 9 or 10, preferably FIG. 8.

In a further embodiment, Form AT4 of Avapritinib may be a solvate.

Yet in a further embodiment, Form AT4 of Avapritinib may be acetic acid solvate.

In a further embodiment, the ratio of Avapritinib:Acetic acid may be 1:1.

In any embodiment, the acetic acid content of Form AT4 of Avapritinib may be in the range of: about 5.5 to about 13 wt %, about 7 to about 12 wt %, about 7.5 to about 11 wt %, about 7.5 to about 10 wt %, or about 10 wt %.

The above crystalline polymorphs can be used to prepare other crystalline polymorphs of Avapritinib, Avapritinib salts and their solid state forms.

Avapritinib Form AT4 may be prepared by crystallization from a solvent comprising acetic acid. Crystallization may be carried out by a process comprising:

(a) preparing a solution of Avapritinib in a solvent comprising acetic acid, and
(b) crystallization of Avapritinib Form AT4 from the solution.
The solvent in step (a) comprises acetic acid, and optionally one or more solvents selected from acetone, ethyl acetate, or water. In embodiments the solution in step (a) comprises, consists essentially of, or is a mixture of, Avapritinib and acetic acid. Alternatively, the solution in step (a) comprises, consists essentially of, or is a mixture of, Avapritinib, acetic acid and water. In embodiments the solution in step (a) comprises, consists essentially of, or is a mixture of Avapritinib, acetic acid, and at least one solvent selected acetone, water, or ethyl acetate. In embodiments, the solvent may be acetic acid, a mixture of acetone and acetic acid, a mixture of acetone, water and acetic acid, a mixture of ethyl acetate and acetic acid, or a mixture water and acetic acid. Preferably, the solution in step (a) comprises, consists essentially of, or is a mixture of Avapritinib, acetic acid, acetone and water. In any embodiment of this process, step (b) may comprise cooling the solution in step (a) or combining the solution in step (a) with an antisolvent. Preferably, step (b) comprises combining the solution in step (a) with an antisolvent. The combining may be carried out at a temperature of about 5° C. to about 30° C., about 8° C. to about 28° C., or about 10° C. to about 25° C. The antisolvent may be an ether, preferably a C₂₋₆ ether, and more preferably methyl tert-butyl ether (MTBE). The antisolvent may be added to the solution, or the solution may be added to the antisolvent (reverse addition). Preferably, the antisolvent is added to the solution. The antisolvent may be added at a temperature of: about 5° C. to about 40° C., about 10° C. to about 35° C., or about 20° C. to about 30° C. Following step (b), the mixture may be stirred, optionally at a temperature of: about 5° C. to about 35° C., about 10° C. to about 30° C., or about 15° C. to about 25° C. for a suitable period of time. The stirring may be conducted over a period of about 30 minutes to about 6 hours, about 30 minutes to about 4 hours, about 45 minutes to about 90 minutes, or about 1 hour. Crystalline Form AT4 may be isolated by any suitable method, including filtration, decantation or centrifuge, preferably by filtration. Crystalline Form AT4 may be dried, optionally in a vacuum oven. The drying may be conducted at about 25° C. to about 80° C., about 30° C. to about 70° C., or about 40° C. to about 65° C. or about 50° C. to about 60° C.

The present disclosure includes a crystalline polymorph of Avapritinib, designated AT5. The crystalline Form AT5 of Avapritinib may be characterized by data selected from one or more of the following: an X-ray powder diffraction pattern substantially as depicted in FIG. 6; an X-ray powder diffraction pattern having peaks at 10.2, 12.1, 14.8, 22.1 and 24.6 degrees 2-theta±0.2 degrees 2-theta; and combinations of these data.

Crystalline Form AT5 of Avapritinib may be further characterized by an X-ray powder diffraction pattern having peaks at 10.2, 12.1, 14.8, 22.1 and 24.6 degrees 2-theta±0.2 degrees 2-theta, and also having any one, two or three additional peaks selected from 3.6, 19.2 and 28.3 degrees 2-theta±0.2 degrees 2-theta.

In one embodiment of the present disclosure, crystalline Form AT5 of Avapritinib is isolated.

In a further embodiment, crystalline Form AT5 of Avapritinib in anhydrous form.

Crystalline Form AT5 of Avapritinib may be characterized by each of the above characteristics alone/or by all possible combinations, e.g., an XRPD pattern having peaks at 10.2, 12.1, 14.8, 22.1 and 24.6 degrees 2-theta±0.2 degrees 2-theta; an XRPD pattern as depicted in FIG. 6, and combinations thereof.

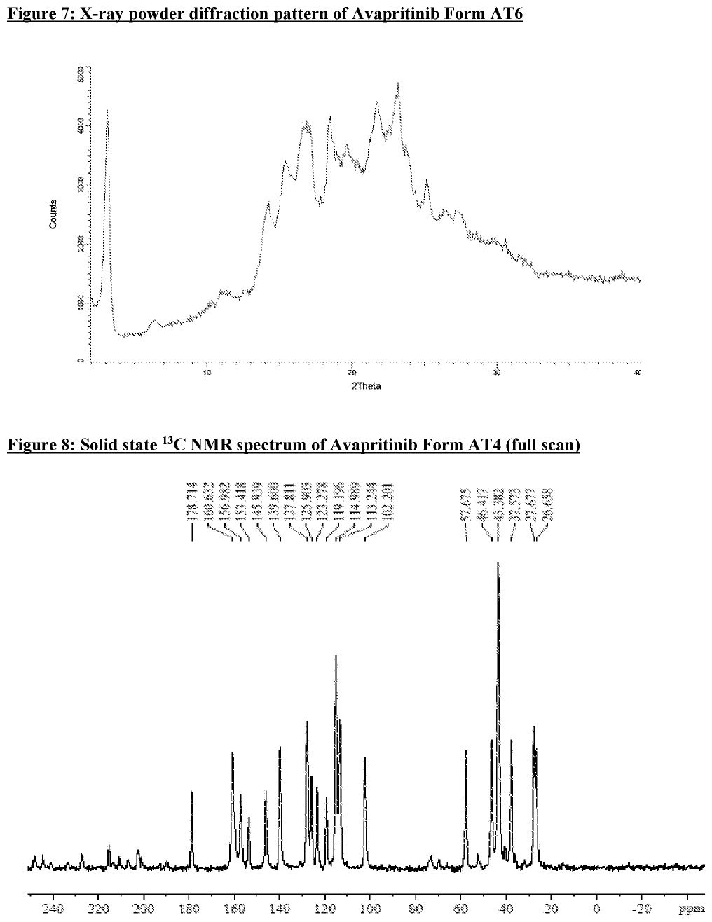

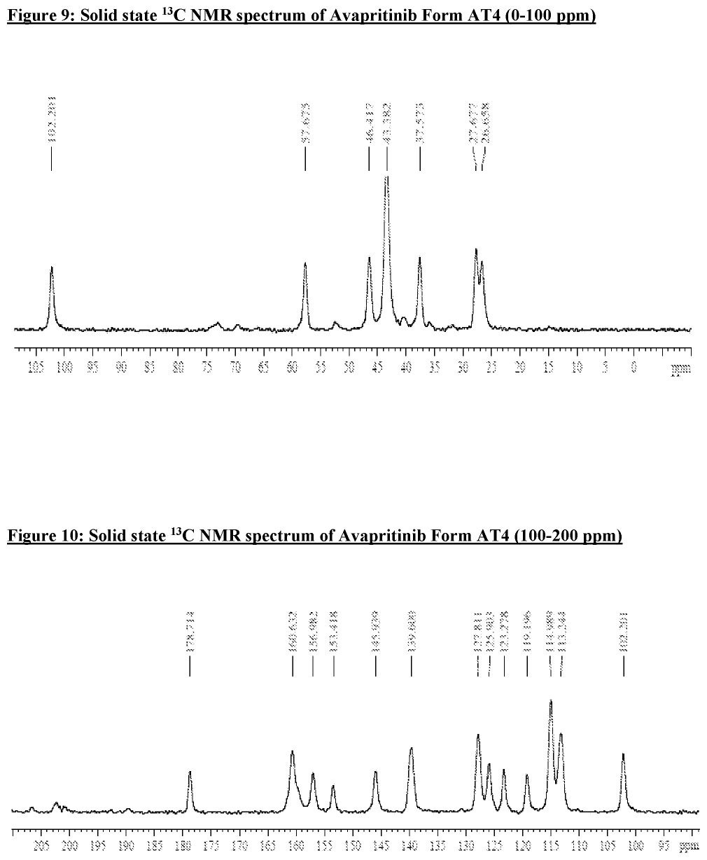

The Present disclosure includes also a crystalline polymorph of Avapritinib, designated AT6. The crystalline Form AT6 of Avapritinib may be characterized by data selected from one or more of the following: an X-ray powder diffraction pattern substantially as depicted in FIG. 7; an X-ray powder diffraction pattern having peaks at 3.2, 18.5, 21.8, 23.2 and 25.3 degrees 2-theta±0.2 degrees 2-theta; and combinations of these data.

Crystalline Form AT6 of Avapritinib may be further characterized by an X-ray powder diffraction pattern having peaks at 3.2, 18.5, 21.8, 23.2 and 25.3 degrees 2-theta±0.2 degrees 2-theta, and also having any one or two additional peaks selected from 14.3 and 15.6 degrees 2-theta±0.2 degrees 2-theta.

In one embodiment of the present disclosure, crystalline Form AT6 of Avapritinib is isolated.

In a further embodiment, Form AT6 of Avapritinib is a solvate.

Yet in a further embodiment, Form AT6 of Avapritinib is tetrahydrofuran (THF) solvate.

The present disclosure encompasses a process for preparing other solid state forms of Avapritinib, Avapritinib salts and solid state forms thereof. The process includes preparing any polymorph according to the present disclosure; or combinations thereof, and converting it to other polymorph of Avapritinib or salt of Avapritinib. The conversion to a salt can be done, for example, by reacting any of the polymorphs of the present disclosure; or combinations thereof, with an appropriate acid, to obtain the corresponding salt.

The present disclosure provides the above described crystalline polymorph of Avapritinib for use in the preparation of pharmaceutical compositions comprising Avapritinib and/or crystalline polymorphs thereof.

The present disclosure also encompasses the use of the crystalline polymorphs of Avapritinib of the present disclosure; or combinations thereof, for the preparation of pharmaceutical compositions of crystalline polymorph Avapritinib and/or crystalline polymorphs thereof.

The present disclosure includes processes for preparing the above mentioned pharmaceutical compositions. The processes include combining any one or a combination of the crystalline polymorphs of Avapritinib of the present disclosure with at least one pharmaceutically acceptable excipient.

Pharmaceutical combinations or formulations of the present disclosure contain any one or a combination of the solid state forms of Avapritinib of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium tri silicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Avapritinib and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Avapritinib can be administered. Avapritinib may be formulated for administration to a mammal, in embodiments to a human, by injection. Avapritinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Avapritinib and the pharmaceutical compositions and/or formulations of Avapritinib of the present disclosure can be used as medicaments, in embodiments in the treatment of gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST).

The present disclosure also provides methods of treating gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST), wherein the method comprises administering a therapeutically effective amount of any one or a combination of the crystalline polymorphs of Avapritinib of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-Ray Diffraction was Performed on X-Ray Powder Diffractometer:

Bruker D8 Advance; CuKα radiation (λ=1.54 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring.

All X-Ray Powder Diffraction peak values are calibrated with regard to standard silicon spiking in the sample.

### SSNMR Method:

Solid-state NMR spectra were measured at 11.7 T using a Bruker Avance III HD 500 US/WB NMR spectrometer (Karlsruhe, Germany, 2013) with 3.2 mm probehead. The ¹³C CP/MAS NMR spectra employing cross-polarization were acquired using the standard pulse scheme at spinning frequency of 11 kHz and a room temperature (300 K). The recycle delay was 8 s and the cross-polarization contact time was 2 ms. The ¹³C scale was referenced to α-glycine (176.03 ppm for ¹³C). Frictional heating of the spinning samples was offset by active cooling, and the temperature calibration was performed with Pb(NO₃)₂. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δυ½ was less than 3.5 Hz at 250 ms of acquisition time).

### EXAMPLES

### Preparation of Starting Materials

Avapritinib can be prepared according to methods known from the literature, for example International Publication No. WO 2015/057873. Avapritinib Form AT1 starting material may be prepared according to any of the processes disclosed herein. Amorphous Avapritinib starting material may be prepared according to Example 3 below.

### Example 1: Preparation of Avapritinib Form AT1

Avapritinib (0.2 g) was dissolved in dichloromethane (3 mL) at 25-30° C. in a test tube. The solution was filtered through 0.45 micron filter. The clear solution was covered with paraffin film with a pinhole and kept for slow solvent evaporation at 15-20° C. After 2 days, the obtained solid was analyzed by XRD and designated as Form AT1; as shown in FIG. 1.

### Example 2: Preparation of Avapritinib Form AT1

Avapritinib (Amorphous (0.1 g)) was taken in a petri-dish and dried in a vacuum oven at 75-80° C. for 8-10 hours. The sample was cooled down to 25-30° C. and analyzed by XRD-Form AT1.

### Example 3: Preparation of Amorphous Avapritinib

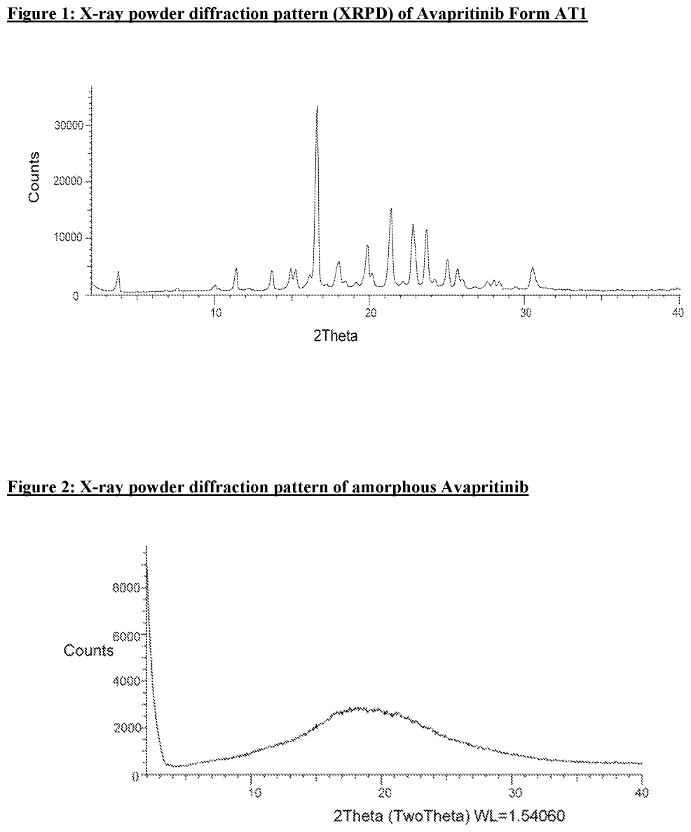

Avapritinib (0.5 g) was charged in a round-bottom flask and slurried in water (10 mL) at 15-25° C. under stirring. Further, dichloromethane (20 ml) was added and stirred for 30 minutes at 15-25° C. to get a clear solution. Aqueous solution of sodium bicarbonate solution (5%) was added to get pH (about) 8. Organic layer was separated and subsequently washed with water (20 ml). The organic layer was distilled out under vacuum at 40-50° C. for 60-120 minutes. The obtained solid (0.42 g) was analyzed by XRD, amorphous form of Avapritinib was obtained; as shown in FIG. 2.

### Example 4: Preparation of Avapritinib Form AT2

Avapritinib (Form AT1, 0.05 g) was taken in a glass vial and ethyl formate (1 ml) was added at 25-30° C. The vial was sealed with silicon septum and maintained under stirring at 25° C. for seven days. The slurry was filtered and the obtained solid was analyzed by XRD—Form AT2; as shown in FIG. 3.

### Example 5: Preparation of Avapritinib Form AT3

Avapritinib (Form AT1, 2.0 g) was dissolved in methanol (200 ml) at 60-65° C. The solution was filtered using 0.45 micron filter. The clear solution was distilled under reduced pressure at 40-50° C. for 1-2 hours. The obtained solid was dried under vacuum at 60° C. and then exposed to 100% relative humidity at 60° C. for 3 days. The obtained solid was analyzed by XRD-Form AT3; as shown in FIG. 4.

### Example 6: Preparation of Avapritinib Form AT4

Avapritinib (AT1, 0.15 g) was dissolved in acetic acid (1 mL) at 30-40° C. The clear solution was cooled to 25° C. The acetic acid solution was slowly added into methyl tert-butyl ether (MTBE, 30 ml) at 25-30° C. under magnetic stirring. The reaction was maintained under stirring for 2-3 hours at 25-30° C. The reaction mixture was filtered, washed with MTBE (5 ml), and dried under vacuum for 30 minutes at 25° C. The wet cake was further dried in vacuum oven at 50° C. for 15 hours. The obtained solid was analyzed by XRD and designated as Form AT4; as shown in FIG. 5.

### Example 7: Preparation of Avapritinib Form AT5

Avapritinib (AT1, 0.5 g) was dissolved in 1,4-Dioxane (10 mL) under magnetic stirring at 55° C. The obtained hot solution was filtered through 0.45 μm PVDF membrane filter and cooled to 25° C. Diisopropyl ether (50 mL) was separately cooled (0-5° C.) and added to the above clear solution at 25° C. The obtained suspension was cooled to 0-5° C. and stirred for 3 hours. The slurry was filtered at 0-5° C. under vacuum for 15 minutes. The obtained solid was analyzed by XRD and designated as Form AT5 of Avapritinib; as shown in FIG. 6.

### Example 8: Preparation of Avapritinib Form AT5

Avapritinib (AT1, 0.05 g) was dissolved in THF (1.25 mL) at 55° C. The obtained hot solution was filtered through 0.45 μm PVDF membrane filter and cooled to 25° C. The clear solution was added to precooled (0-5° C.) n-heptane (3.75 mL) at 5° C. and the obtained suspension was stirred at 0-5° C. for 3 hours. The slurry was filtered at 0-5° C. under vacuum for 15 minutes. The obtained solid was analyzed—Form AT5.

### Example 9: Preparation of Avapritinib Form AT5

Avapritinib (AT1, 0.05 g) was dissolved in THF (1.25 mL) at 55° C. The obtained hot solution was filtered through 0.45 μm PVDF membrane filter and cooled to 25° C. The clear solution was added to precooled (0-5° C.) diisopropyl ether (3.75 mL) at 5° C. The obtained suspension was stirred at 0-5° C. for 3 hours and filtered under vacuum (at 0-5° C.) for 15 minutes. The obtained solid was analyzed by XRD-Form AT5.

### Example 10: Preparation of Avapritinib Form AT6

Avapritinib (AT3, 0.03 g) was dissolved in tetrahydrofuran (0.2 mL) at 45-50° C. and added methyl tert-butyl ether (1 ml) at 25° C. The reaction mixture was maintained under stirring for 4 hours at 25° C. The reaction mass was filtered under vacuum at 20-25° C. for 30 minutes. The obtained solid was analyzed by XRD and designated as Form AT6 of Avapritinib; as shown in FIG. 7.

### Example 11: Preparation of Avapritinib Form AT1

Avapritinib (5 g) was dissolved in dichloromethane (100 ml) at 25-30° C. The obtained solution was filtered through Whatman 42 filter paper. The obtained solution was distilled under vacuum at 40-45° C. for 1-2 hours. The obtained solid was analyzed by XRD and designated as Form AT1 of Avapritinib.

### Example 12: Preparation of Avapritinib Form AT4

In a reactor, (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine (0.2 g), acetone (1 mL) and acetic acid (0.14 mL) were charged at 20-30° C. followed by stirring at 20-30° C. Methyl tert-butyl ether (3 mL) was added at 20-30° C. followed by stirring for 1 hour at 20-30° C. Solid was filtered and dried under vacuum at 50-60° C. to get (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine. The obtained solid was analyzed by XRD and designated as Avapritinib Form AT4.

### Example 13: Preparation of Avapritinib Form AT4

In a reactor, (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine (0.2 g), acetone (1 mL) and acetic acid (0.4 mL) were charged at 20-30° C. followed by stirring at 20-30° C. Methyl tert-butyl ether (3 mL) was added at 20-30° C. followed by stirring for 1 hour at 20-30° C. Solid was filtered and dried under vacuum at 50-60° C. to get (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine. The obtained solid was analyzed by XRD and designated as Avapritinib Form AT4.

### Example 14: Preparation of Avapritinib Form AT4

In a reactor, (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine (0.15 g), aqueous acetone (5% water) (1 ml) and acetic acid (0.15 mL) were charged at 20-30° C. followed by stirring at 20-30° C. Methyl tert-butyl ether (3 mL) was added at 20-30° C. followed by stirring for 1 hour at 20-30° C. Solid was filtered and dried under vacuum at 50-60° C. to get (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine. The obtained solid was analyzed by XRD and designated as Avapritinib Form AT4.

### Example 15: Preparation of Avapritinib Form AT4

In a reactor, (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine (0.15 g), ethyl acetate (1 ml) and acetic acid (0.15 mL) were charged at 20-30° C. followed by stirring at 20-30° C. Methyl tert-butyl ether (3 mL) was added at 20-30° C. followed by stirring for 1 hour at 20-30° C. Solid was filtered and dried under vacuum at 50-60° C. to get (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine. The obtained solid was analyzed by XRD and designated as Avapritinib Form AT4.

### Example 16: Preparation of Avapritinib Form AT4

In a reactor, (S)-1-(4-fluorophenyl)-1-(2-(4-(6-(1-methyl-1H-pyrazol yl)pyrrolo[2,1-f][1,2,4]triazin-4-yl)piperazin-1-yl)pyrimidin-5-yl)ethan-1-amine (0.15 g), acetone (1 ml) and acetic acid (0.15 mL) were charged at 10-15° C. followed by stirring at 10-15° C. Methyl tert-butyl ether (3 mL) was added at 20-30° C. followed by stirring for 1 hour at 20-30° C. Solid was filtered and dried under vacuum at 50-60° C. to get (S)-1-(4-fluorophenyl)-1-(2-(4-(6-(1-methyl-1H-pyrazol-4-yl)pyrrolo[2,1-f][1,2,4]triazin-4-yl)piperazin-1-yl)pyrimidin-5-yl)ethan-1-amine. The obtained solid was analyzed by XRD and designated as Avapritinib Form AT4.

1. A crystalline form of Avapritinib, designated Form AT4, which is characterized by data selected from one or more of the following:
(a) an X-ray powder diffraction pattern substantially as depicted in FIG. 5;
(b) an X-ray powder diffraction pattern having peaks at 11.3, 16.3, 21.3, 22.9 and 23.9 degrees 2-theta±0.2 degrees 2-theta;
(c) a solid state ¹³C NMR spectrum with peaks at 27.7, 57.7, 113.2, 123.3, 139.6 and 178.7 ppm±0.2 ppm;
(d) a solid state ¹³C NMR spectrum having the following chemical shift absolute differences from a peak at 43.4 ppm±2 ppm of 15.7, 14.3, 69.8, 79.9, 96.2 and 135.3 ppm±0.1 ppm;
(e) a solid state ¹³C NMR spectrum substantially as depicted in any of FIG. 8, 9 or 10;
and combinations of these data.

2. Crystalline Avapritinib according to claim 1, which is characterized by data selected from one or more of the following:
(a) an X-ray powder diffraction pattern substantially as depicted in FIG. 5;
(b) an X-ray powder diffraction pattern having peaks at 11.3, 16.3, 21.3, 22.9 and 23.9 degrees 2-theta±0.2 degrees 2-theta; or a combination thereof.

3. Crystalline Avapritinib according to claim 2, which is further characterized by a solid state ¹³C NMR spectrum with peaks at 27.7, 57.7, 113.2, 123.3, 139.6 and 178.7 ppm±0.2 ppm.

4. Crystalline Avapritinib according to claim 2, which is further characterized by a solid state ¹³C NMR spectrum having the following chemical shift absolute differences from a peak at 43.4 ppm±2 ppm of 15.7, 14.3, 69.8, 79.9, 96.2 and 135.3 ppm±0.1 ppm.

5. Crystalline Avapritinib according to claim 2, which is further characterized by a solid state ¹³C NMR spectrum substantially as depicted in any of FIG. 8, 9, or 10.

6. Crystalline Avapritinib according to claim 2, which is further characterized by an X-ray powder diffraction pattern having any one, two, three or four additional peaks selected from 9.5, 14.3, 15.9, and 16.9 degrees 2-theta±0.2 degrees 2-theta.

7. Crystalline Avapritinib according to claim 1, which is characterized by an XRPD pattern having peaks at 9.5, 11.3, 14.3, 15.9, 16.3, 16.9, 21.3, 22.9 and 23.9 degrees 2-theta±0.2 degrees 2-theta.

8. (canceled)

9. (canceled)

10. Crystalline Avapritinib according to claim 1, which is polymorphically pure.

11. Crystalline Avapritinib according to claim 1, which is substantially free of any other solid state forms of Avapritinib.

12. Crystalline Avapritinib according to claim 1, containing about 20% (w/w) or less of any other forms of Avapritinib.

13. (canceled)

14. (canceled)

15. A pharmaceutical composition comprising crystalline Avapritinib according to claim 1, and at least one pharmaceutically acceptable excipient.

16. (canceled)

17. A process for preparing a pharmaceutical composition comprising combining crystalline Avapritinib according to claim 1 with at least one pharmaceutically acceptable excipient.

18. A medicament comprising the Crystalline Avapritinib according to claim 1.

19. (canceled)

20. A method of treating gastrointestinal stromal tumors (GIST), solid tumors, or Advanced Systemic Mastocytosis, comprising administering a therapeutically effective amount of the crystalline Avapritinib according to claim 1 to a subject in need of treatment.
